(19) United States
(12) Patent Application Publication
(10) Pub. No.: US 2026/0141977 A1
(43) Pub. Date: May 21, 2026

(54) PROTEIN DESIGN TOOLS TO MINIMIZE THE CAS PROTEIN SIZE WHILE MAINTAINING ITS FUNCTION

(71) Applicant: THE UNIVERSITY OF NORTH TEXAS HEALTH SCIENCE CENTER, Ft. Worth, TX (US)

(72) Inventors: Jin Liu, Dallas, TX (US); Vindi M. Jayasinghe-Arachchige, White Settlement, TX (US)

(21) Appl. No.: 19/392,718

(22) Filed: Nov. 18, 2025

Related U.S. Application Data
(60) Provisional application No. 63/721,888, filed on Nov. 18, 2024.

(51) Int. Cl. G16B 15/20; C12N 9/22; G16B 15/30; G16B 30/10; G16B 40/20

(52) U.S. Cl. CPC G16B 15/20; C12N 9/226; G16B 15/30; G16B 30/10; G16B 40/20

(57) ABSTRACT

The large-sized CRISPR Cas proteins have hindered the effective use of CRISPR-mediated gene editing in human therapeutics due to the lack of delivery strategies for these large-sized proteins to reach their target locations. As the use of highly targeted medicine increases, health care related business and individuals seek additional ways to more efficiently develop personalized medicine. CRISPR technologies revolutionized the gene therapy field in the past decade. AI-based tools to optimize the size of Cas proteins as well as developed gene-editing tools of experimentally-validated artificial-designed mini-Cas proteins were developed; thereby, shifting the paradigm of CRISPR research field from searching for new Cas proteins in nature to designing artificial Cas using AI algorithms.

### CROSS REFERENCE TO RELATED APPLICATIONS

This application claims priority to U.S. Prov. App. 63/721,888 filed Nov. 18, 2024, which is incorporated by reference herein in its entirety.

### GOVERNMENT RIGHTS

This invention was made with government support under GM144860 awarded by the National Institutes of Health. The government has certain rights in the invention.

### FIELD OF THE DISCLOSURE

The present disclosure generally relates to CRISPR-mediated gene editing.

### BACKGROUND

The large-sized CRISPR Cas proteins have hindered the effective use of CRISPR-mediated gene editing in human therapeutics due to the lack of delivery strategies for these large-sized proteins to reach their target locations.

### SUMMARY

One of the bottlenecks for advancing CRISPR technology into clinical settings is the delivery of the large-sized CRISPR-Cas proteins to their target site. Multiple efforts have been taken to improve the CRISPR delivery, including searching for Cas with the smaller size, designing large nanoparticles as the carrier, etc. Though Artificial Intelligence programs are improving all the time, no protein engineering research has been reported to optimize the size of proteins or to design artificial mini-Cas proteins using AI algorithms.

Shortcomings mentioned here are only representative and are included simply to highlight that a need exists for improved verification of information. Embodiments described herein address certain shortcomings but not necessarily each and every one described here or known in the art. Furthermore, embodiments described herein may present other benefits than, and be used in other applications than, those of the shortcomings described above.

Various strategies have been explored to enhance CRISPR delivery, such as identifying smaller Cas proteins, developing large nanoparticles as carriers, etc. However, no protein engineering research has been reported to optimize the size of proteins or to design artificial mini-Cas proteins using AI algorithms. This application discloses tools using the cutting-edge AI technologies to optimize the size of Cas proteins and tested the feasibility of the tools by developing experimental validated artificial-designed mini-Cas proteins. In doing so, embodiments of this disclosure make available tools to optimize the size of proteins using the cutting-edge AI technologies as well as deliver the first artificial signed Cas protein validated in biochemical and cell-based assays addressing the CRISPR delivery challenge. As a result, the paradigm of CRISPR research field has shifted from searching for new Cas proteins in nature to designing artificial Cas using AI algorithms.

Embodiments of this disclosure include AI-based tools to optimize the size of Cas proteins as well as developed gene-editing tools of experimentally-validated artificial-designed mini-Cas proteins. Recently, the mini-Cas protein CasΦ, which is about the half size of the most commonly-used Cas9 and Cas12a, has been discovered in phages with the double-strand DNA cleavage activity, implying that smaller protein may have the potential to serve as gene-editing tools. Embodiments of this disclosure include various artificial mini-Cas proteins with guided double-stranded DNA cleavage function designed using AI-based tools to optimize Cas proteins. In this effort, Cas-design technology was developed using two alternative approaches. Although two approaches are described below, embodiments of this disclosure may be considered to include approaches implementing portions of individually-disclosed approaches and combinations thereof.

The first approach centered on developing Cas-design technology using sequence-based generative adversarial network and attention-based neural network. Focusing on the design of novel Cas sequences, multiple sequence alignment (MSA) and co-evolutionary analyses were used to train a classification model. The generative adversarial network and attention-based neural networks was used to generate new sequences with a minimized number of amino acids. The classification model was used to predict the guided double DNA cleavage capability of the generated sequences. Currently available protein structure prediction software such as, AlphaFold, I-Tassel, and trRosetta was used to predict the 3D structure from the sequence and evaluate protein structure stability. The stable structures with the minimum number of amino acids was simulated by molecular dynamics simulations for further evaluation. The most promising candidate was synthesized and evaluated in biochemical and cell-based assays.

The second approach is centered on developing Cas-design technology using structure-based reinforcement learning algorithm through semi-unsupervised learning. The sequence design step was skipped, and design proceeded directly from 3D protein structures. Drawing on the principles behind MuZero and AlphaZero, two AI programs that achieved superhuman performance in the unrelated and challenging Go game through self-play, protein design is conceptualized as a ‘protein optimizing’ game. Protein structures were treated as connected network graphs, and a graph representation learning model was developed to learn high-dimensional continuous encodings of protein. To reduce the conformational space and achieve efficient machine learning, two reinforcement learning based approaches to discover mini-Cas proteins were developed. First generated “seed” residues, used in Cas functions, were identified using MSA and co-evolutionary analyses.

Based on the “seed” residues, the first approach is a “growing game”. The process began with the 3D positions of the “seed” residues, which were used to “grow” a protein. The residues in between “seed” residue pairs in the 3D space were trained using the 3D structures in the protein data bank until the minimum number of residues is reached with a stable structure. The second approach is a “removing game”. Starting with CasΦ, the smallest known Cas protein reinforcement learning was used to remove residues located between the identified seed residues. This process continued until a stable mini-Cas protein structure was achieved. The computational and experimental validations were similar to the first approach.

Two approaches were developed to adapt the AI algorithms used in protein structure prediction and strategic board games to develop tools to minimize the Cas protein size. This advancement in protein design provided tools to minimize the size of design proteins. The lack of tools to solve the minimize size of design proteins in a specific manner is a problem that had never been solved. The first artificially designed Cas proteins validated in biochemical and cell-based assays greatly advanced the CRISPR field by addressing the CRISPR delivery problem and shifting the paradigm of the CRISPR research field from searching for new Cas proteins from nature to designing artificial Cas using AI algorithms.

### A. Applications/Uses of the Disclosure

At least one impact of the disclosure has led to advancing biological research and clinical application of CRISPR-Cas technology by providing experimentally-validated artificial-designed mini-Cas protein tools. Mini-Cas proteins were designed to significantly improve the delivery of the CRISPR-Cas systems and create a new horizon to develop therapeutic strategies for many diseases that require in vivo CRISPR-Cas delivery. Also, the min-Cas proteins provide a variety of new gene-editing tools in basic biological studies, including generating cell lines to investigate functions of portions of the genome or pathologies, transgenic animals with deletions in a certain cell type, and other biomedical studies.

At least one impact of the disclosure has led to advancing the technical field of protein design by providing tools to optimize the size of enzymes using cutting-edge AI technologies. Indeed, the fundamental problem about the possible minimum size of a protein has not been solved yet. This disclosure has solved this problem that has not been solved before using cutting-edge AI technologies.

At least one impact of the disclosure has led to the opening of new research avenues to design artificial Cas using AI algorithms. Considering the wide application of the CRISPR-Cas system in more and more fields, including biomedical, agriculture, biofuels, etc., there is an increased need for a versatile toolbox of CRISPR-Cas proteins. Our work has opened new research avenues and shifted the paradigm of the current approaches of “searching new Cas from nature” to “designing artificial Cas in the lab”.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF DRAWINGS

It will be appreciated that for simplicity and clarity of illustration, elements illustrated in the Figures have not necessarily been drawn to scale. For example, the dimensions of some of the elements are exaggerated relative to other elements. Embodiments incorporating teachings of the present disclosure are shown and described with respect to the drawings presented herein, in which:

FIG. 1 is a block diagram of an example set of objectives and milestones to develop mini-Cas protein using AI technologies according to some embodiments of the disclosure.

FIG. 2 is a block diagram of an example method for creating an AI tool to develop mini-Cas protein according to some embodiments of the disclosure.

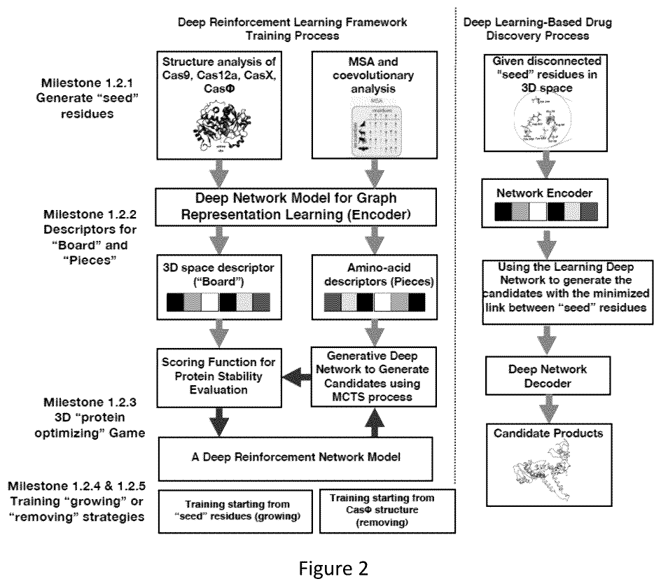

FIG. 3 is a diagram of an example method for creating the Monte Carlo tree search according to some embodiments of the disclosure.

### DETAILED DESCRIPTION

The following description in combination with the Figures is provided to assist in understanding the teachings disclosed herein. The following discussion will focus on specific implementations and embodiments of the teachings. This focus is provided to assist in describing the teachings and should not be interpreted as a limitation on the scope or applicability of the teachings. However, other teachings can certainly be used in this application. The teachings can also be used in other applications and with several different types of architectures.

### A. Example Description of Terms

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Further, the words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “some embodiments,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present embodiment. Thus, the appearances of the foregoing phrases in various places throughout this specification are not necessarily all referring to the same embodiment. Furthermore, the particular features, structures, or characteristics may be combined in any suitable manner In some embodiments.

### B. General Embodiments

### 1. Technological Hypothesis and Specific Aims

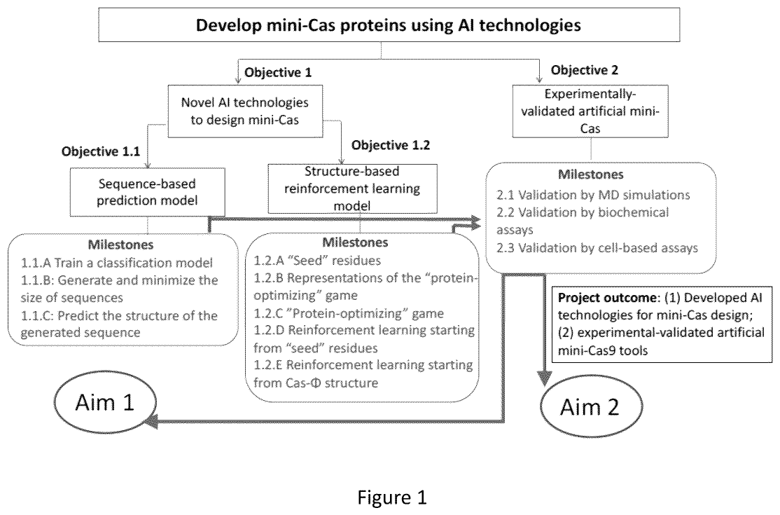

Using AI technologies, artificial mini-Cas proteins with guided double-stranded DNA cleavage function were designed. This was achieve by developing the Cas-design using two alternative approaches. The flowchart of the design process is shown in FIG. 1.

The first approach centers on developing Cas-design technology using the sequence-based generative adversarial network and attention-based neural network. The design of novel Cas sequences by using the combined generative adversarial network and attention-based neural network was focused on. Multiple sequence alignment (MSA) and distance constraints from amino acid residue co-evolution to train a classification model were used. The generative adversarial network and attention-based neural network are used to generate new sequences with a minimized number of amino acids. The classification model is used to predict the guided double DNA cleavage capability of the generated sequences. The protein structure prediction software comprising AlphaFold, I-Tassel, and trRosetta are used to predict the 3D structure from the sequence and evaluate protein structure stability. The stable structures with the minimum number of amino acids are simulated by molecular dynamics simulations for further evaluation. The most promising candidates are synthesized and evaluated in biochemical and cell-based assays.

The second approach centers on developing Cas-design technology using structure based reinforcement learning algorithm through semi-unsupervised learning. The sequence design was skipped and design proceeded directly from structure. The protein design task as a “protein optimizing” game within the AI was set up. Protein structures are treated as connected network graphs, and graph representation learning is applied to learn high dimensional continuous encodings of protein. The learned continuous encoding representation represent the conformational space the computer program learns and explore for new protein designs. To reduce the conformational space, two alternative approaches were used. “Seed” residues, which are used in Cas functions, were first generated are then identified using MSA and co-evolutionary analyses. The first approach is a “growing game”. The design process begins with the 3D positions of the “seed” residues, which are used to “grow” a protein. The residues in between “seed” residue pairs in the 3D space are trained using the 3D structures in the protein data bank until the minimum number of residues are reached with a stable structure. The second approach is a “removing game”. The design process starts with the currently known smallest Cas, CasΦ, and proceeds by removing the residues in between the “seed” residues with unsupervised learning. The computational and experimental validations are similar to the first approach.

### 2. Objectives and Milestones

Objective 1 centers on developing AI-based tools to optimize the size of Cas proteins.

Objective 1.1 is achieved by developing sequence-based prediction model using the generative adversarial network and attention-based neural network.

Milestone 1.1.A, training a classification model, is achieved by using approximately 1.8 million endonuclease sequences and approximately 10k endonuclease structural data to perform MSA and co-evolutionary analysis, similar to AlphaFold. Together with the Cas sequences with PAM recognition and guided double-stranded DNA cleavage activities, a classification model was trained to predict if a given sequence can be classified into the Cas sequence.

Milestone 1.1.B, generating and minimizing the size of sequences, is achieved by generating the endonuclease sequence using two published approaches, the attention-base neural network and the generative adversarial network. The codes of these two published works were customized for use in the AI. The classification model generated in milestone 1.1.A is used to determine if the generated sequence could be classified as the Cas sequence. The lengths of the current available double-stranded DNA cleavage Cas are ˜700-1400 amino acids (aa) and the target protein sequence length is below 300 aa. To avoid the bias of the training set, sequences were generated with the gradually reduced sequence lengths; thereby, training the model with gradient reduced size.

Milestone 1.1.C, predicting the structure of the generated sequence, is achieved by implementing customized versions of the AlphaFold codes into the AI enabling prediction of the structure of the generated sequence. I-Tasser and trRosetta are used to cross-validate the predicted structures. Further validations are described in Objective 2.

Objective 1.2 is achieved by developing a model-based reinforcement learning model through semi-unsupervised learning in 3D space.

Milestone 1.2.A, generating “seed” residues, is achieved by the MSA, co-evolutionary analysis, and structural analysis that is performed for the species of Cas with PAM recognition and guided double stranded DNA cleavage activities. The “seed” residues are determined at least based on the conserved and/or co-evolved PAM recognition, guide RNA binding, DNA binding, and/or DNA cleavage sites.

Milestone 1.2.B, developing the representations of the “protein-optimizing” game is achieved by graph representation learning to generate the game “board” and “pieces”. Most strategy board games played by humans are in a two dimensional (2D) space, apparently limited by the human perception capability. This, however, is not an issue for artificial intelligence, as AI is able to search high dimensional space including three-dimensional (3D) space. A protein structure including amino acid position, chemical bond, bond angle, and dihedral angles are certainly not continuous and are discretized in the 3D space. Therefore, a 3D discretization method was developed to divide space into numerous 3D compartments to host amino acid residues. These 3D compartments provide a template or a 3D board game to build new amino acids.

A new generative model of molecular graphs using VAE was developed. While one could imagine solving the problem in a standard manner—generating graphs node by node—the approach is not ideal for protein structures. This is because creating protein atom by atom would force the model to generate chemically invalid intermediaries, delaying validation until a complete graph is generated. Instead, protein graphs in two phases by exploiting valid subgraphs as components were generated. The overall generative approach, cast as a junction tree variational autoencoder, first generates a tree-structured object (a junction tree) residue node whose role is to represent the scaffold of subgraph components and their coarse relative arrangements. The components are valid chemical substructures automatically extracted from the training set using tree decomposition and are used as building blocks. In the second phase, the subgraphs (nodes in the tree) are assembled into a coherent protein residue graph. The original protein residue node graph and its associated junction tree offer two complementary representations of a protein residue. The protein residues are encoded into a two-part latent representation z=[zT, zG] where zT encodes the tree structure and what the clusters are in the tree without fully capturing how exactly the clusters are mutually connected; zG encodes the graph to capture the fine-grained connectivity. Both parts are created by tree and graph encoders q(zT|T) and q(zG|G). The latent representation is then decoded back into a molecular graph in two stages. First, the junction tree was reproduced using a tree decoder p(T|zT) based on the information in zT. Second, the fine grain connectivity between the clusters in the junction tree is predicted using a graph decoder p(G|T, zG) to realize the full protein graph. The junction tree approach maintains chemical feasibility during generation.

Milestone 1.2.C, developing the 3D strategy “protein-optimizing” game by adapting AlphaZero as a generative model of protein structure.

Implement reinforcement learning algorithms: AlphaZero does not learn from human players' data but from the process of self-play. It evaluates each step it plays using a deep neural network and updates parameters in the neural network after a certain time of self-play or so-called iteration. That is a typical process of reinforcement learning.

Policy iteration is a classic algorithm that generates a sequence of improving policies, by alternating between policy evaluation—estimating the value function of the current policy—and policy improvement—using the current value function to generate a better policy. A simple approach to policy evaluation is to estimate the value function from the outcomes of sampled trajectories. A simple approach of policy improvement is to select actions greedily with respect to the value function. In large state spaces, approximations are necessary to evaluate each policy and to represent its improvement.

The deep neural network used in AlphaZero are denoted as a mapping function f_{θ}with parameters θ. This neural network takes board position s as the input and outputs both reaction probabilities ‘ρ’ and a value ‘ν’ estimating the expected outcome, which is represented as (ρ, ν)=f_{θ}(s). The vector of move probabilities ρ represents the probability of selecting each candidate reactant, ρ_{α}=Pr (a|s). The value ν is a scalar evaluation, estimating the probability of the current reactant leading to current compound s. This neural network combines the roles of both policy network and value network into a single architecture. The neural network consists of many residual blocks of convolutional layers with batch normalization and rectifier nonlinearities. The neural network in AlphaGo Zero is trained from games of self-play by a novel reinforcement learning algorithm. In each position, a Monte Carlo tree search (MCTS) algorithm is executed, guided by the neural network f_{θ}(s). The MCTS algorithm outputted probabilities π of using each reactant. These search probabilities usually selected much stronger reactants than the raw move probabilities ρ of the neural network f_{θ}(s); MCTS are, therefore, viewed as a powerful policy improvement operator. Self-play with search—using the improved MCTS-based policy to select each reactant, then using the game-winner z as a sample of the drug—are a powerful policy evaluation operator. The main idea of the reinforcement learning algorithm is to use MCTS-based search operators repeatedly in the policy iteration procedure, such that the neural network's parameters are updated to make the move probabilities and value (ρ, ν)=f_{θ}(s) more closely match the improved search probabilities and the self-play winner (π, z). The updated new parameters are used in the next iteration of self-play to make the next search step even stronger.

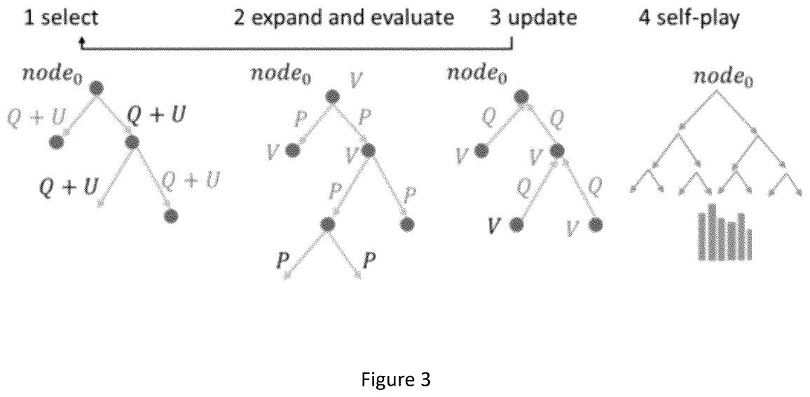

Implement MCTS: The MCTS uses the neural network f_{θ}to guide its simulations (see FIG. 3). Each edge (s, a) in the search tree stores a prior probability P(s, a), a visit count N(s, a), and an action value Q(s, a). Each simulation started from the root state and iteratively selected reactants that maximize an upper confidence bound Q(s, a)+U(s, a), where U(s, a)∝P(s, a)/(1+N(s, a)), until a leaf nodes' was encountered. This leaf position is expanded and evaluated once by the network to generate both prior probabilities and evaluation, (P(s′, ⋅), V(s′))=f_{θ}(s′). Each edge (s, a) traversed in the simulation is updated to increment its visit count N(s, a), and to update its action value to the mean evaluation over these simulations, Q(s, a)=1/N(s, a)Σ_{s′|α→s′}V(s′), where s′|α→s′ indicates that a simulation eventually reached s′ after taking move a from position s. MCTS, viewed as a self-play algorithm that, given neural network parameters θ and a root position s, computes a vector of search probabilities recommending interactions to produce, π=α_{θ}(s), proportional to the exponentiated visit count for each reactant, π_{α}∝ N(s, a)1/τ, where τ is a temperature parameter.

Milestone 1.2.D, developing reinforcement learning by starting from “seed” residues. This is a “growing” game. The “seed” residues determined in Milestone 1.2.A are placed in 3D space at the start of the game. A search over the PDB database was completed to determine the shortest path between “seed” residues to “grow” the smallest protein. The scoring function is used to evaluate the stabilities of the protein and compare it with a certain threshold to determine whether AI “wins” the game and thereby discovering a stable protein structure. This process repeats until AI wins the game.

Milestone 1.2.E, developing reinforcement learning starting from Cas-Φ structure. This is a “removing” game. The smallest known double-stranded cleavage Cas, Cas-Φ (˜700 amino acid residues), was used at the start of the game. The residues in between the “seed” residues were removed and replaced with candidates of shorter “seed” connection sequences using reinforcement learning. The scoring function is used to evaluate the stabilities of the generated protein and compare it with a certain threshold to determine whether AI “wins” the game. This process repeats until AI wins the game.

Objectives 2 centered on developing gene-editing tools of experimental-validated artificial mini-Cas proteins.

Milestone 2.1, validating the stability and function of designed top candidates using molecular dynamics (MD) simulations. Generated Amino acid sequences design candidates determined to be highly stable with an effective double-stranded DNA cleavage capability are then evaluated in a simulation. MD simulations of the design candidates were performed to further evaluate the stabilities of these designed Cas proteins. DNA and gRNA are docked into the system and the complex is simulated to evaluate the binding affinities. The top candidates are then subjected to validation in biochemical and cell-based assays.

Milestone 2.2, validating the design proteins with protein synthesis and evaluate double-stranded DNA cleavage activities using biochemical assays. Genes coding for the selected design proteins are synthesized. The plasmids are purchased from Genscript. Completely sequence confirmed positive clones are transformed into Escherichia coli Rosetta strain 2(DE 3 ) for recombinant protein expression and protein is purified using established protocols. Standardized protocols yielding high amounts of pure gRNA using in vitro transcription and purification methods were also used.

Milestone 2.3, validating the double-stranded DNA cleavage activities in cells using cell-based assays. Cell-based assay to assess the biological function of CRISPR-Cas proteins candidates was developed. Plasmids/genetic constructs are created to express CRISPR in E. coli cells that are expected to target a fluorescent reporter gene. Activities of CRISPR-Cas candidates for removing the reporter gene are assessed with a flow cytometric method. A screening platform to be used to test at least 200 CRISPR-Cas protein candidates on a daily basis was developed. A range of candidates designed by the AI technologies and use their developed platform to assay their activities to determine if the designed Cas has double-stranded DNA cleavage activities in cells was also constructed.

Engineered proteins generated through AI design can be subjected to purification. The method can include introducing a gene encoding the target protein into an appropriate expression system, producing the protein in a host cell, and applying purification steps to isolate the desired product. The process can employ sequential operations intended to improve protein yield and quality for use in downstream applications. In certain embodiments, chromatographic techniques are utilized to achieve these enhancements. These approaches can provide flexibility in optimizing purity and functionality while maintaining the structural integrity of the engineered protein. In some cases, the purification protocol may be adapted to accommodate different protein variants or host systems. Such variations allow the method to be broadly applicable across multiple contexts without departing from the scope of the invention.

In some embodiments, a purification method yields the artificial mini-Cas protein with an amount of purity and/or enzymatic activity reaching 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### ADDITIONAL RECITED EMBODIMENTS:

Embodiment 1: A method, comprising: training an artificial intelligence (AI) model using multiple sequency alignment (MSA) and co-evolutionary analyses based on an initial set of endonuclease sequences; and determining, using the artificial intelligence (AI) model, an artificial mini-Cas protein with guided double-stranded DNA cleavage function, wherein the artificial mini-Cas protein has a size smaller than a predetermined size.

Embodiment 2: The method of embodiment 1, wherein the artificial mini-Cas protein predetermined size has a size smaller than half of Cas9 or Cas12a.

Embodiment 3: The method of embodiment 1, wherein the artificial intelligence (AI) model comprises at least one of an attention-based neural network or a generative adversarial network model.

Embodiment 4: The method of embodiment 1, wherein the training comprises reinforcement learning model or graph-based representation learning model.

Embodiment 5: The method of embodiment 1, wherein determining the artificial min-Cas protein comprises: determining a sequence using a sequence-based prediction model; determining a structure corresponding to the sequence; and training the artificial intelligence (AI) model based on the structure.

Embodiment 6: The method of embodiment 5, wherein determining the sequence comprises: determining an endonuclease sequence with a sequence length less than an average sequence length of the initial set of endonuclease sequences; determining if the endonuclease sequence can be classified as a Cas sequence; and updating training of the artificial intelligence (AI) model based on the endonuclease sequence to reduce an average sequence length of the artificial intelligence (AI) model.

Embodiment 7: The method of embodiment 5, wherein determining the endonuclease sequence comprises: generating at least one tree-structured object residue node whose role is to represent the scaffold of subgraph components and their coarse relative arrangements, wherein the subgraph components comprise valid chemical substructures; assembling the at least one nodes in the tree into a coherent protein residue graph; encoding the protein residue graph into a two-part latent representation comprising zT encoding the tree structure and zG encoding the graph; and decoding the two-part latent representation into a molecular graph.

Embodiment 8: An apparatus, comprising: a memory configured to store an artificial intelligence (AI) model; and at least one processor coupled to the memory and configured to perform the steps recited in embodiments 1-7.

Embodiment 9: A method, comprising: training, an amino acid sequence generator artificial intelligence (AI), using sequence based adversarial network AI and attention-based neural network AI based on an initial set of double-stranded DNA cleavage Cas; and determining, using the amino acid sequence generator AI, an artificial mini-Cas protein with guided double-stranded DNA cleavage function, wherein the artificial mini-Cas protein has a size less than threshold value.

Embodiment 10: The method of embodiment 9, sequence-based prediction model comprises: training a classification AI using multiple sequency alignment (MSA) and co-evolutionary analyses based on an initial set of endonuclease sequences; training an amino acid structure predictor AI, using AlphFold, I-Tassel, tr-Rosetta based on an initial set of endonuclease sequences; determining, using the amino acid sequence generator AI, an artificial mini-Cas protein with guided double-stranded DNA cleavage function; and determining, using the Classification AI, the guided double-stranded DNA cleavage capability rating of the artificial mini-Cas protein.

Embodiment 11: The method of embodiment 10, wherein the cleavage capability rating is greater than a threshold value, and the stability rating is greater than a threshold value.

Embodiment 12: The method of embodiment 9, wherein the initial set of double-stranded DNA cleave Cas is between 700-1400 amino acids.

Embodiment 13: The method of embodiment 9, wherein the artificial mini-Cas protein size threshold value is no higher than 300 amino acids.

Embodiment 14: The method of embodiment 10, wherein the cleavage rating is at least in the top 10% of cleavage ratings.

Embodiment 15: The method of embodiment 10, wherein the stability rating is at least in the top 10% of stability ratings.

Embodiment 16: A method, comprising: training, an amino acid sequence generator artificial intelligence (AI), using sequence based adversarial network AI and attention-based neural network AI based on an initial set of double-stranded DNA cleavage Cas; training a classification AI using multiple sequency alignment (MSA) and co-evolutionary analyses based on an initial set of endonuclease sequences; training an amino acid structure predictor AI, using AlphFold, I-Tassel, tr-Rosetta based on an initial set of endonuclease sequences; determining, using the amino acid sequence generator AI, an artificial mini-Cas protein with guided double-stranded DNA cleavage function; determining, using the Classification AI, the guided double-stranded DNA cleavage capability rating of the artificial mini-Cas protein; determining, using the amnio acid structure predictor AI, a 3D structure and stability rating of the artificial mini-Cas protein; and wherein the artificial mini-Cas protein size is less than a threshold value, the cleavage capability rating is greater than a threshold value, and the stability rating is greater than a threshold value.

Embodiment 17: The method of embodiment 16, wherein the initial set of double-stranded DNA cleave Cas is between 700-1400 amino acids.

Embodiment 18: The method of embodiment 16, wherein the artificial mini-Cas protein size threshold value is no higher than 300 amino acids.

Embodiment 19: The method of embodiment 17, wherein the cleavage rating is at least in the top 10% of cleavage ratings.

Embodiment 20: The method of embodiment 18, wherein the stability rating is at least in the top 10% of stability ratings.

Embodiment 21: A method, comprising: receiving, more than one disconnected seed residue in 3D space; encoding the more than one disconnected seed residue in 3D space for use in a Learning Deep Network AI; determining, using the Learning Deep Network AI, a candidate product with least links between seed residues; decoding the candidate product with the least links between seed residues;

Embodiment 22: The method of embodiment 21, wherein the Learning Deep Network AI is trained according to a method comprising: determining, using the Classification AI, the guided double-stranded DNA cleavage capability rating of the artificial mini-Cas protein; determining, using the amnio acid structure predictor AI, a 3D structure and stability rating of the artificial mini-Cas protein; and wherein the cleavage capability rating is greater than a threshold value, and the stability rating is greater than a threshold value.

What is claimed:

1. A method, comprising:
training an artificial intelligence (AI) model using multiple sequency alignment (MSA) and co-evolutionary analyses based on an initial set of endonuclease sequences; and
determining, using the artificial intelligence (AI) model, an artificial mini-Cas protein with guided double-stranded DNA cleavage function, wherein the artificial mini-Cas protein has a size smaller than a predetermined size.

2. The method of claim 1, wherein the artificial mini-Cas protein predetermined size has a size smaller than half of Cas9 or Cas12a.

3. The method of claim 1, wherein the artificial intelligence (AI) model comprises at least one of an attention-based neural network or a generative adversarial network model.

4. The method of claim 1, wherein the training comprises reinforcement learning model or graph-based representation learning model.

5. The method of claim 1, wherein determining the artificial min-Cas protein comprises:
determining a sequence using a sequence-based prediction model;
determining a structure corresponding to the sequence; and
training the artificial intelligence (AI) model based on the structure.

6. The method of claim 5, wherein determining the sequence comprises:
determining an endonuclease sequence with a sequence length less than an average sequence length of the initial set of endonuclease sequences;
determining if the endonuclease sequence can be classified as a Cas sequence; and
updating training of the artificial intelligence (AI) model based on the endonuclease sequence to reduce an average sequence length of the artificial intelligence (AI) model.

7. The method of claim 5, wherein determining the endonuclease sequence comprises:
generating at least one tree-structured object residue node whose role is to represent the scaffold of subgraph components and their coarse relative arrangements, wherein the subgraph components comprise valid chemical substructures;
assembling the at least one nodes in the tree into a coherent protein residue graph;
encoding the protein residue graph into a two-part latent representation comprising zT encoding the tree structure and zG encoding the graph; and
decoding the two-part latent representation into a molecular graph.

8. An apparatus, comprising:
a memory configured to store an artificial intelligence (AI) model; and
at least one processor coupled to the memory and configured to perform the steps recited in claims 1-7.

9. A method, comprising:
training, an amino acid sequence generator artificial intelligence (AI), using sequence based adversarial network AI and attention-based neural network AI based on an initial set of double-stranded DNA cleavage Cas; and
determining, using the amino acid sequence generator AI, an artificial mini-Cas protein with guided double-stranded DNA cleavage function, wherein the artificial mini-Cas protein has a size less than threshold value.

10. The method of claim 9, wherein the sequence-based prediction model comprises:
training a classification AI using multiple sequency alignment (MSA) and co-evolutionary analyses based on an initial set of endonuclease sequences;
training an amino acid structure predictor AI, using AlphFold, I-Tassel, tr-Rosetta based on an initial set of endonuclease sequences;
determining, using the amino acid sequence generator AI, an artificial mini-Cas protein with guided double-stranded DNA cleavage function; and
determining, using the Classification AI, the guided double-stranded DNA cleavage capability rating of the artificial mini-Cas protein.

11. The method of claim 10, wherein the cleavage capability rating is greater than a threshold value, and the stability rating is greater than a threshold value.

12. The method of claim 9, wherein the initial set of double-stranded DNA cleave Cas is between 700-1400 amino acids.

13. The method of claim 9, wherein the artificial mini-Cas protein size threshold value is no higher than 300 amino acids.

14. The method of claim 10, wherein the cleavage rating is at least in the top 10% of cleavage ratings.

15. The method of claim 10, wherein the stability rating is at least in the top 10% of stability ratings.

16. A method, comprising:
training, an amino acid sequence generator artificial intelligence (AI), using sequence based adversarial network AI and attention-based neural network AI based on an initial set of double-stranded DNA cleavage Cas;
training a classification AI using multiple sequency alignment (MSA) and co-evolutionary analyses based on an initial set of endonuclease sequences;
training an amino acid structure predictor AI, using AlphFold, I-Tassel, tr-Rosetta based on an initial set of endonuclease sequences;
determining, using the amino acid sequence generator AI, an artificial mini-Cas protein with guided double-stranded DNA cleavage function;
determining, using the Classification AI, the guided double-stranded DNA cleavage capability rating of the artificial mini-Cas protein;
determining, using the amnio acid structure predictor AI, a 3D structure and stability rating of the artificial mini-Cas protein; and
wherein the artificial mini-Cas protein size is less than a threshold value, the cleavage capability rating is greater than a threshold value, and the stability rating is greater than a threshold value.

17. The method of claim 16, wherein the initial set of double-stranded DNA cleave Cas is between 700-1400 amino acids.

18. The method of claim 16, wherein the artificial mini-Cas protein size threshold value is no higher than 300 amino acids.

19. The method of claim 17, wherein the cleavage rating is at least in the top 10% of cleavage ratings.

20. The method of claim 18, wherein the stability rating is at least in the top 10% of stability ratings.

21. A method, comprising:
receiving, more than one disconnected seed residue in 3D space;
encoding the more than one disconnected seed residue in 3D space for use in a Learning Deep Network AI;
determining, using the Learning Deep Network AI, a candidate product with least links between seed residues;
decoding the candidate product with the least links between seed residues.

22. The method of claim 21, wherein the Learning Deep Network AI is trained according to a method comprising:
determining, using the Classification AI, the guided double-stranded DNA cleavage capability rating of the artificial mini-Cas protein;
determining, using the amnio acid structure predictor AI, a 3D structure and stability rating of the artificial mini-Cas protein; and
wherein the cleavage capability rating is greater than a threshold value, and the stability rating is greater than a threshold value.
